(19) United States
(12) Patent Application Publication
(10) Pub. No.: US 2017/0128595 A1
(43) Pub. Date: May 11, 2017

(54) MICROBIAL GROWTH INDICATING MEDICAL DEVICES

(71) Applicant: Indicator Systems International, Inc., Newport Beach, CA (US)

(72) Inventors: Gerald F. Swiss, Rancho Santa Fe, CA (US); Robert M. Moriarty, Michiana Shores, MI (US); Ram W. Sabnis, Atlanta, GA (US)

(73) Assignee: Indicator Systems International, Inc., Newport Beach, CA (US)

(21) Appl. No.: 15/321,726

(22) PCT Filed: Jun. 22, 2015
(86) PCT No.: PCT/US2015/037031

§ 371 Date: Dec. 22, 2016

Related U.S. Application Data
(63) Continuation of application No. 14/312,541, filed on Jun. 23, 2014, now abandoned.
Application No. 14/312,541 is a continuation-in-part of application No. PCT/US2014/037211, filed on May 7, 2014.
(60) Provisional application No. 61/821,616, filed on May 9, 2013.

(51) Int. Cl. A61K 49/00

(52) U.S. Cl. CPC A61K 49/0043

(57) ABSTRACT

Provided herein are medical devices capable of self-reporting microbial growth adjacent the site of the implanted device.

### FIELD OF THE INVENTION

This invention provides for the use of pH dependent fluorescent molecules to determine the presence of an incipient microbial infection in vivo. Such molecules can be used topically in combination with wound dressings or in implantable medical devices. In either case, these molecules are capable of self-reporting microbial growth adjacent thereto. Further, the invention provides for non-invasive methods to self-report the microbes at a site having or suspected of microbial growth.

### BACKGROUND OF THE INVENTION

Infections at the site of implantation of a medical device are a serious problem. For example, surgeries relating to breast implants result in infection rates from about 2% to as high as 20% in women undergoing such implants, with the highest rate of infection in reconstructive cases. Feldman, et al., Plast. Reconstr. Surg., 126(3): 779-85 (2010). Similarly, prosthetic joint infections are a frequent cause of prosthesis failure. Gemmel, et al., Eur. J. Nucl. Med. Mol. Imaging, 39(5):892-909 (2012). A variety of bacteria and fungi may be involved in such infections, with staphylococci, including Staphylococcus epidermidis and S. aureus, accounting for a majority of infections.

Frequently, infection of the site of implantation of a medical device requires that the device be removed and/or replaced. This results in increased risk to the patient as well as increased cost. In addition, infection can lead to serious illness, and even death, if the infection is unnoticed and untreated for even a relatively short period of time. Undetected bacterial infection may result in sepsis, septic phlebitis, septic shock, bacteraemia, tunnel infection, and/or metastatic complications (e.g., endocarditis, osteomyelitis, or septic thrombosis). Accordingly, early detection of bacterial infection in the region of the implantation site of a medical device is highly desirable.

Therefore, a need exists for methods and medical devices for the early detection of bacterial growth at or around the implantation site of a medical device that can readily detect and indicate the presence of microorganisms well before the infection has progressed to the point that it manifests itself by clinical symptoms.

### SUMMARY OF THE INVENTION

This invention is related to the discovery that in vivo microbial, such as bacterial, growth and infections, such as those related to the implantation of a medical device will alter the pH of the environment at and near the infection. Specifically, physiologic fluid has a pH from about 7 to about 7.3. The presence of an active microbial infection will result in production of carbon dioxide and other components which, when mixed with physiological fluid, convert to acidic components such as carbonic acid. The presence of carbonic acid and other acidic components are detectable by self-identifying indicators. These self-identifying indicators produce a differential signal due to the pH change which signal can be assessed ex vivo to ascertain the presence of an infection in a patient.

Accordingly, in one embodiment, this invention is directed to medical devices comprising on at least part of their surface self-identifying indicators which indicators produce or can be induced to produce a differential signal under acidic pH as compared to the signal produced at neutral or alkaline pH wherein said signal can be assessed ex vivo. The medical devices can comprise implanted or topical devices such as artificial joints, intravenous catheters, pace makers, sutures, wound coverings, and the like.

In one embodiment, the self-identifying indicators can be pH-dependent liposomes which comprise a paramagnetic ion under neutral or alkaline pH. In another embodiment, these indicators are pH sensitive dyes which change structure and hence alter at least one of their electromagnetic emission characteristics in going from an alkaline or neutral pH to an acidic pH. In yet another embodiment, these indicators are pH sensitive fluorescent indicators.

In one of its method aspects, this invention provides for an ex vivo method to determine the presence of microbial growth at or adjacent to a medical device implanted on or in a patient which method comprises

selecting a medical device having on at least part of its surface self-identifying indicators which indicators produce a differential signal under acidic pH as compared to the signal produced at neutral or alkaline pH wherein said signal can be assessed ex vivo,

placing said medical device on or in a patient;

monitoring ex vivo the signal produced by the self-identifying indicators; and

correlating the signal so produced to the presence or absence of microbial growth.

In one embodiment, the medical devices contain self-identifying reporters either by themselves or in combination with the indicators set forth above. Such reporters include compounds bound to the antibody or binding fragment thereof and which emit a differential signal when bound to the microbe as compared to that when not bound to the microbe. For example, such a differential signal can be a signal arising from a change in at least one electromagnetic emission character of the reporter when bound as opposed to when not bound to the microbe.

In yet another embodiment, these reporters are fluorescent indicators which have an altered fluorescence when bound to the microbe as compared to being unbound.

In another embodiment, the medical device contains on at least part of its surface an antibody or binding fragment thereof which specifically binds to a microbe and produces a signal indicating the identity of the microbe bound thereto. In some embodiments, the antibody or binding fragment thereof has bound thereto a reporter, such as a fluorescent moiety which changes its fluorescent character upon binding to the microbe. In some embodiments, a plurality of different antibodies or binding fragments thereof are bound to the medical device, each producing a unique signal for the microbe bound thereto.

Also provided herein are ex vivo methods to determine the microbe present in an infection at or adjacent to a medical device implanted in a patient which method comprises

selecting an implantable medical device having on at least part of its surface self-identifying reporters which reporters produce a differential signal when bound to a microbe as compared to the signal produced when not bound to a microbe wherein said signal can be assessed ex vivo,

placing said medical device in a patient;

monitoring ex vivo the signal produced by the self-identifying reports; and

correlating the signal so produced to the presence or absence of the microbe at an active infection.

In one embodiment, the signal produced by the indicator and/or reporter on the implanted medical device is measured immediately after implantation and that signal is used as a baseline or reference signal for comparison to future signals so as to aid the clinician in determining the degree of change in the emitted signal or signals.

In some embodiments, the method further comprises treating a patient with antimicrobial compounds, such as antibiotics.

### BRIEF DESCRIPTION OF THE DRAWINGS

FIG. 1 is the absorbance spectrum of heptamethoxy red at neutral pH.

FIG. 2 is the absorbance spectrum of heptamethoxy red at an acidic pH.

### DETAILED DESCRIPTION OF THE INVENTION

This invention provides for medical devices capable of self-identifying the presence of microbial growth at or adjacent the medical device and/or the infecting microbe. However, prior to discussing this invention in detail, the following terms will first be defined.

### DEFINITIONS

It must be noted that, as used in the specification and any appended claims, the singular forms “a,” “an,” and “the” include plural referents unless the context clearly dictates otherwise. Similarly, the plural forms include singular referents unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

As used herein, an “implantable medical device” refers to any type of medical device that is totally or partly introduced, surgically or medically, into a patient's body or by medical intervention into a natural orifice, temporarily or for a period of time. In some aspects, the medical device is intended to be removed during or upon completion of the procedure. In some aspects, the medical device is intended to remain there after the procedure. The duration of implantation may be between transient, such as for the duration sufficient to retrieve a sample form the patient's body, and essentially permanent, i.e., intended to remain in place for the lifespan of the product or patient; or until it is physically removed or biodegrades. Examples of essentially permanent implantable medical devices include, without limitation, implantable cardiac pacemakers and defibrillators; leads and electrodes for the preceding; implantable organ stimulators such as nerve, bladder, sphincter and diaphragm stimulators; cochlear implants; prostheses, including artificial knees, hips, and other joint replacements; vascular grafts, self-expandable stents, balloon-expandable stents, stent-grafts, grafts, artificial heart valves, cerebrospinal fluid shunts; renal dialysis shunts; artificial hearts; implantable infusion pumps; breast implants; dental implants; surgical mesh; and implantable access systems. Examples of implantable devices also include topical devices such as bandages, wraps and tapes that are applied to skin wounds. In some embodiments of the skin wound, at least a section of the skin surface layer, the epidermis, which is naturally acidic, is wounded or damaged, such that the exposed skin inner layer or tissue has a physiological pH of above 7 absent any microbial growth.

Alternatively, the duration of the implant may be temporary or transient. That is to say that the implant is intended to remain in place for a defined period of time which, however, is sufficient to allow an infection to develop. Temporary implants may be inserted from 1 day through 2 years or longer. Examples of temporary implants include sutures, catheters, intravenous injection ports, braces, and the like. Examples of transient implantable medical devices include, without limitation, syringes whose tip can be introduced into a patient's body or a natural orifice and catheters. In some embodiments, the tip of the syringe or catheter comprises indicators attached or incorporated thereto and a removable cap. The cap covers the area of the tip having the indicators and insulates the tip from outside environment so that it is not contaminated with materials, such as physiological fluid or tissue, that are not to be tested. The cap can be opened, such as by pushing the tip, when the tip is placed at a location where possible microbial growth is to be detected, and closed after a sample is retrieved by the tip to protect the tip and the sample from contamination when device is withdrawn from the location being tested. The absence or presence of microbial growth can be determined by detection of the signals produced by the indicators on the tip with or without removing the cap.

The term “surface” as it relates to the implantable medical device refers to the outer surface of the device interfacing with physiological fluid and tissue or organs of a patient. For example, both the exterior and the interior walls of the lumen of a catheter are outer surfaces of the catheter as both the exterior and the interior walls can be in contact with a physiological fluid or tissue when used in a medical procedure. Similarly, the interior wall of the cartridge of a syringe is an outer surface of the syringe as the interior wall can be in contact with a physiological fluid or tissue. As described below, in a preferred embodiment, the surface of the medical device comprises a surface layer to which an indicator or reporter has been integrated therein or can be attached by post-treatment to from covalent linkages thereto. In an embodiment, the surface comprises a mesh or similar covering, for example the surgical mesh pouches disclosed in PCT Pub. No. WO 2008/127411. In yet another embodiment, the surface comprises a biodegradable or bioerodable layer which covers and thereby protects the indicators and/or reporters during implantation. In such an embodiment, the surface of the medical device constitutes three components—the inner component defining the surface of the medical device; an intermediate component which comprises a plurality of indicators and/or reporters bound to the medical device surface; and an outer component which is a biodegradable or bioerodable layer forming the outer surface.

The term “biodegradable or bioerodable layer” refers to a biocompatible material which degrades or erodes in vivo to expose the underlying surface. Such materials can be any material well known in the art which provides for an outer coating on the device. For example, biodegradable materials include hyaluronic acid, collagen, polylactides, polyglycolides, polycaprolactones, polydioxanones, polycarbonates, polyhydroxybutyrates, polyalkylene oxalates, polyanhydrides, polyamides, polyesteramides, polyurethanes, polyacetals, polyketals, polyorthocarbonates, polyphosphazenes, polyhydroxyvalerates, polyalkylene succinates, poly(malic acid), poly(amino acids), chitin, chitosan, and polyorthoesters, and copolymers, terpolymers and combinations and mixtures thereof. See, for example, Dunn, et al., U.S. Pat. No. 4,938,763 which is incorporated herein by reference in its entirety.

The term “patient” refers to any mammalian patient and includes without limitation primates such as humans, monkeys, apes, and the like, and domesticated animals such as horses, dogs, cats, ovines, bovines, and the like.

As used herein, the term “indicator” refers to a compound or device that produces a signal in presence of microbial growth. The signal can be electromagnetic such as a change in absorption which can be observed by naked eye and/or by using an emission and/or absorption spectrophotometer. Such indicators include by way of example only, dyes including pH indicators, metals such as gadolinium, pH sensitive fluorescent indicators and the like.

Suitable pH indicators include, by way of example only, phenol red, xylenol blue, bromocresol purple, bromocresol green, Congo red, cresol red, phenolphthalein, bromothymol blue, p-naphtholbenzein, neutral red, a mixture of potassium iodide, mercuric (III) iodide, sodium borate, sodium hydroxide and water nile blue, thymolphthalein, crysol violet, hydroxy naphthol blue, malachite green oxalate, methyl orange, alizarin, crystal violet, methyl red, fluorescein, and derivatives and mixtures thereof as well as food grade dyes provided that in each case such indicators generate a signal when in the presence of microbial growth. Suitable pH sensitive fluorescent indicators include, but not limited to, 6,7-dihydroxy-4-methylcoumarin, 7-hydroxycoumarin and derivatives thereof, which are non-fluorescent in acidic pH and become blue fluorescent toward neutral pH. The structures of these is given below:

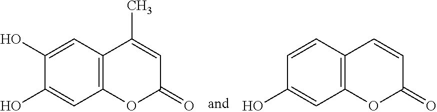

In some embodiments, the indicator described herein is a metal selected from the group of a fluorescent moiety; a paramagnetic ion, such as gadolinium, europium, manganese, lanthanide, iron, and derivatives thereof; or a phase transition material. The indicator is capable of remote detection, for example by magnetic resonance imaging (MRI). Examples of these and other indicators are well-known in the art. For example, and without limiting the scope of the present invention, Amanlou, et al. describes several indicators that are commonly used in MRI, including small mononuclear or polynuclear paramagnetic chelates; metalloporphyrins; polymeric or macromolecular carriers (covalently or noncovalently labeled with paramagnetic chelates); particulate contrast agents (including fluorinated or non-fluorinated paramagnetic micelles or liposomes) and paramagnetic or super paramagnetic particles (e.g., iron oxides, Gd3-labeled zeolites); dimagnetic CEST polymers; dimagnetic hyperpolarization probes (gases and aerosols), and 13C-labeled compounds or ions. Amanlou, et al., Current Radiopharmaceuticals 4, 31-43 (2011).

In some embodiments, the indicator is pH-sensitive or temperature-sensitive.

In an embodiment, the indicator is a fluorescent moiety. Fluorescence is the light emitted when a molecule absorbs light at a higher energy wavelength and emits that light at a lower energy wavelength. In an embodiment, the fluorescent moiety is remotely detectable, for example by fluorescence spectroscopy.

In an embodiment, the fluorescent moiety is present in a liposome at self-quenching concentration. In an embodiment, a liposome comprises the fluorescent moiety and a fluorescent quencher.

In an embodiment, the indicator is fluorescein or a derivative thereof, or a salt of fluorescein or the derivative.

Fluorescein is of the formula:

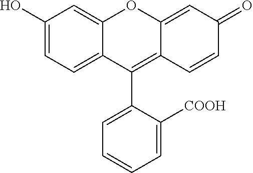 [SMILES: O=C(O)c1ccccc1-c1c2ccc(=O)cc-2oc2cc(O)ccc12]

or a tautomeric structure.

Salts of fluorescein or a derivative include, but are not limited to, the sodium salt and disodium salt, potassium salt and dipotassium salt.

Peak excitation of fluorescein occurs at 494 nm and peak emission at 521 nm. The absorption and fluorescence of fluorescein or its derivatives are pH dependent. For example, fluorescein has a pKa of 6.4, and its ionization equilibrium leads to pH-dependent absorption and emission over the range of 5 to 9. Extinction coefficients and fluorescence quantum of fluorescein yields decrease at pH<7, such as pH 5.5. The fluorescence lifetimes of the protonated and deprotonated forms of fluorescein are approximately 3 and 4 ns, which allows for pH determination from nonintensity based measurements. Determination of pH according to the absorption and emission of fluorescein is known in the art, see, e.g. Joseph R. Lakowicz, Principles of Fluorescence Spectroscopy, 638-639 (3rd ed. 2006).

In some embodiments, the fluorescein derivative is of the formula:

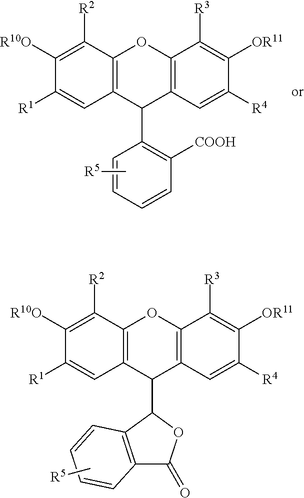

or a tautomer therefore or a pharmaceutically acceptable salt of the compound or tautomer, wherein
R¹, R², R³ and R⁴ are independently selected from the group consisting of hydrogen, fluoro, chloro, bromo, and —O—C₁-C₄ alkyl,
R⁵ is hydrogen, COOH or —(C(O)NH)ₘ(CH₂))ₙ—COOH, m is 0 or 1, n is 1, 2, 3, 4, or 5; and
   R¹⁰ and R¹¹ are independently hydrogen or —C(O)C₁-C₄ alkyl.

In some embodiments, R² and R³ are hydrogen.

In some embodiments, R¹⁰ and R¹¹ are hydrogen.

Examples of fluorescein derivatives include but are not limited to, 2′,7′-dichlorofluorescein, 5(6)-carboxyfluorescein, 5(6)-carboxyfluorescein diacetate, 5-carboxyfluorescein, 6-[fluorescein-5(6)-carboxamido]hexanoic acid, 6-carboxyfluorescein, fluorescein diacetate 5-maleimide, fluorescein-O′-acetic acid which are available from Sigma-Aldrich Co., Missouri, USA. Additional fluorescein derivatives include 2′,7′-difluorofluorescein (OREGON GREEN™)

Use of the fluorescent indicators in the detection of pH changes is known in the art and is described in, for example, Junyan Han and Kevin Burgess, Fluorescent Indicators for Intracellular pH, Chem. Rev., 2010, 110(5):2709-2728, which is incorporated herein by reference in its entirety.

In an embodiment, the indicator is sensitive to pH. pH-sensitive indicators are described, for example, in U.S. Patent Pub. No. 2011/0104261 A1 and references therein, all of which are incorporated herein by reference in their entirety. Such pH-sensitive indicators may include citraconyl-linked Gd chelates, Gd diethylenetriamine pentaacetic acid (DTPA) chelates, and Gd-DOTA chelates.

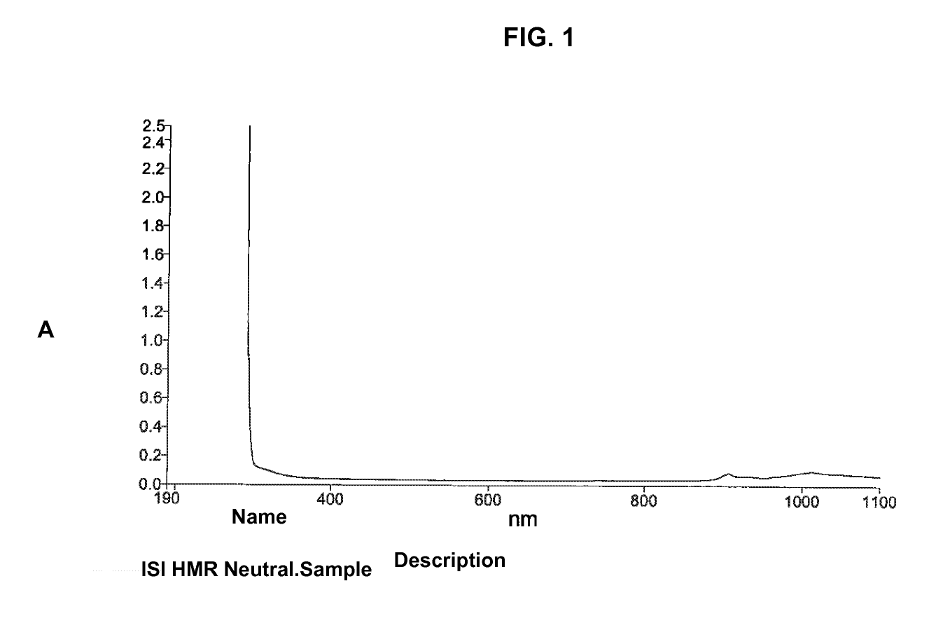

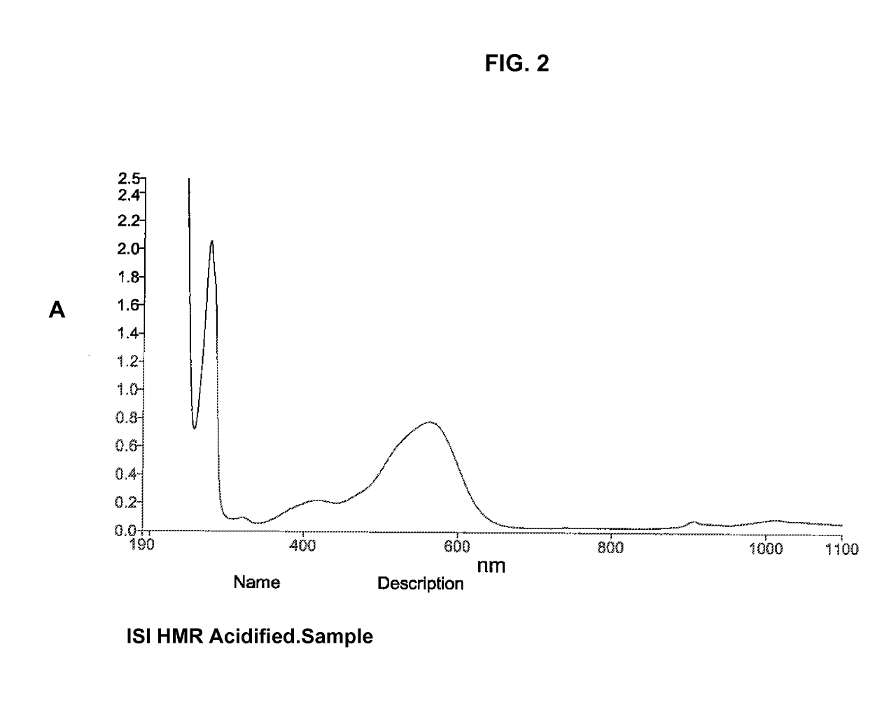

In a preferred embodiment, the fluorescent moiety is heptamethoxy red (HMR). The absorbance of light by HMR in a non-acidic environment is different compared to that of acidic HMR. Specifically, acidic HMR has absorbance in the blue light range whereas neutral HMR does not. See FIGS. 1 and 2 which illustrate this differential absorption pattern. In turn, this allows for detection of which form of the HMR exists in a given sample and, in a physiological fluid, allows for ascertaining whether that fluid is acidic or not as well as the degree of acidity.

In some embodiments, the fluorescent moiety and the indicator are identical. In other embodiments, the fluorescent moiety and the indicator are different. In addition, fluorescent pH indicators are well known in the art and some of which are commercially available. In a preferred embodiment, such fluorescent pH indicators can sense pH changes within physiological ranges. See, for example, http://www.invitrogen.com/site/us/en/home/References/Molecular-Probes-The-Handbook/pH-Indicators/Overview-of-pH-Indicators.html

In some embodiments, the indicator is covalently bound to at least a portion of the surface of the device. In another embodiment, the indicator is integrated at least into or on a portion of the surface layer of the device.

In some embodiments, the indicators utilized herein are selected from hexamethoxy red, heptamethoxy red and derivatives thereof. Methods of making hexamethoxy red and heptamethoxy red are well known to the skilled artisan. See, for example, U.S. Pat. No. 8,425,996, which is incorporated herein in its entirety by reference. Derivatives of hexamethoxy red and heptamethoxy red, including those that are capable of covalently binding to a compatible functional group on the surface of the medical device, and methods of their synthesis are disclosed in U.S. patent application Ser. No. 13/715,014, which is incorporated herein in its entirety by reference.

Specifically, hexamethoxy red and heptamethoxy red can be synthesized following art recognized methods with the appropriate substitution of commercially available reagents as needed. Other compounds are synthesized following modifications of the methods illustrated herein, and those known, based on this disclosure. See, for example, Raj. B. Durairaj, Resorcinol: Chemistry, Technology, and Applications, Birkhauser, 2005. Illustrative and non-limiting methods for synthesizing such compounds are schematically shown below which show the synthesis of an intermediate 4-hydroxyphenyl compound. That compound is subsequently modified on the hydroxyl group to incorporate the polymerizable group or to attach to at least a portion of the surface of a device.

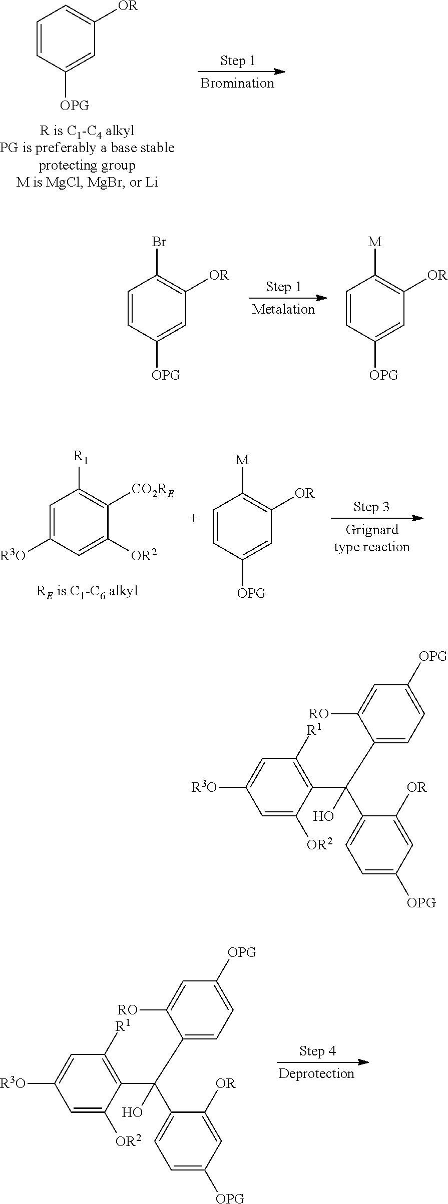

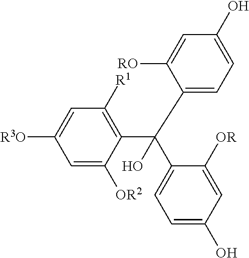

In step 1, a protected resorcinol methyl ether is brominated, preferably using 1 equivalent of bromine in a non-polar solvent such as dioxane. As used herein, PG refers to a protecting group, which refers to well known functional groups that, when bound to a functional group, render the resulting protected functional group inert to the reaction to be conducted on other portions of the compound and the corresponding reaction condition, and which can be reacted to regenerate the original functionality under deprotection conditions. Examples of protecting groups useful for synthesizing the compounds of this invention, and methods for protection and deprotection employed herein, are found in standard reference works such as Greene and Wuts, Protective Groups in Organic Synthesis., 2d Ed., 1991, John Wiley & Sons, and McOmie, Protective Groups in Organic Chemistry, 1975, Plenum Press. Methylthiomethyl ether and allyl ethers are certain non-limiting protecting groups contemplated for the scheme above. In step 2, the brominated resorcinol derivative is metalated to provide a Grignard reagent or a resorcyl lithium. In step 3, the metalated aryl is reacted with an aryl carboxylic acid ester to provide a protected precursor to the compound of Formula (I), which is deprotected in step 4.

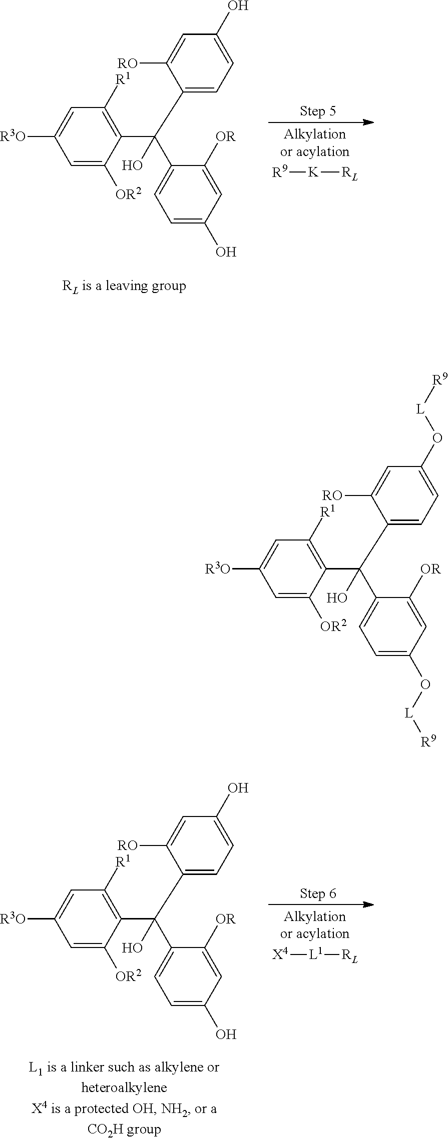

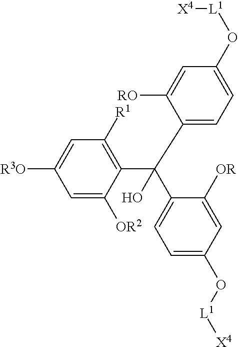

In step 5, the deprotected phenolic hydroxy compound is reacted with an R⁹-L moiety containing a leaving group such as chloro, bromo, iodo, or —OSO₂R_{S} where R_{S} is C₁-C₆ alkyl optionally substituted with 1-5 fluoro atoms or aryl optionally substituted with 1-3 C₁-C₆ alkyl or halo groups. Alternatively, the deprotected compound is reacted with a compound that provides part of the linker L (step 6). Such compounds can be elaborated as shown in steps 7 and 8 below using reagents and methods well known to the skilled artisan.

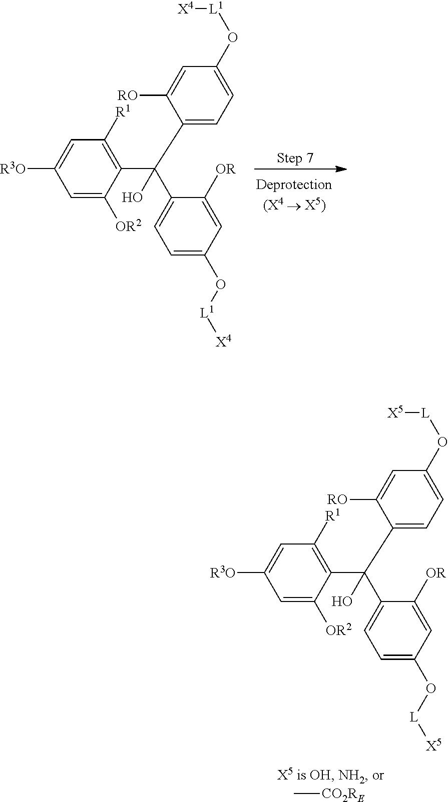

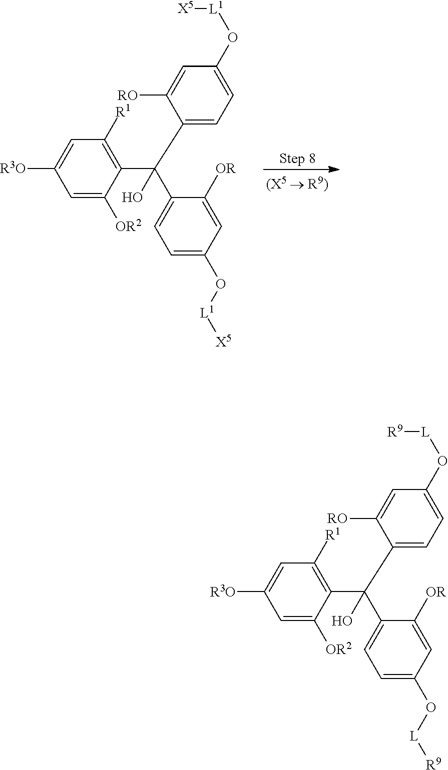

These compounds are also synthesized by reacting an appropriately protected aryl carboxylic acid ester with the metalated aryl compound and elaborating the triaryl methyl compounds produced, via methods provided herein and/or via methods well known to the skilled artisan.

These compounds can also be synthesized by reacting an appropriately protected aryl carboxylic acid ester with the metalated aryl compound and elaborating the triaryl methyl compounds produced, via methods provided herein and/or via methods well known to the skilled artisan:

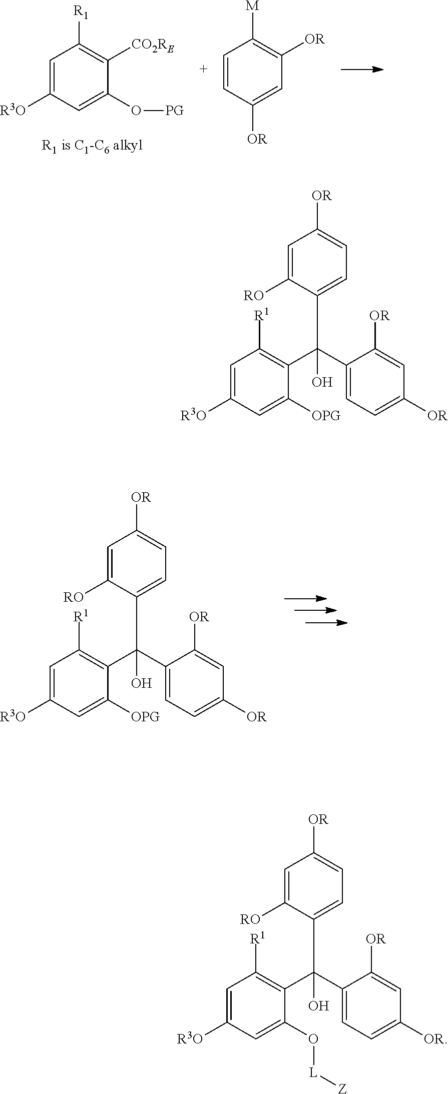

Other compounds for use herein are conveniently synthesized following these and other known methods upon appropriate substitution of starting material and, if needed, protecting groups. Electron withdrawing substituents such as halo can be conveniently incorporated into the aryl rings by electrophilic substitution employing hypohalite, halogens, ICl, preferably under alkaline conditions. A halo group is conveniently converted to a cyano group following well known methods, such as those employing CuCN. A nitro group is conveniently incorporated by electrophilic nitration employing various conditions and reagents well known to the skilled artisan, such as nitronium tetrafluoroborate, nitric acid, optionally with acetic anhydride, and the like.

Other compounds can be prepared following methods well known to a skilled artisan and/or those disclosed herein upon appropriate substitution of reactants and reagents.

Preferred compounds for use herein include those represented below:

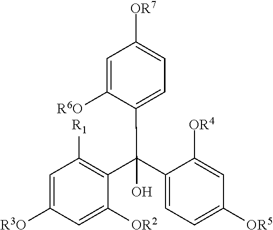

TABLE 1

| Ex. | | R², | | | |
|---|---|---|---|---|---|
| No. | R¹ | R⁴, R⁶ | R³ | R⁵ | R⁷ |
| 1 | H | —CH₃ | —CH₃ | —CH₃ | —CH₂CH═CH₂ |
| 2 | H | —CH₃ | —CH₂CH═CH₂ | —CH₃ | —CH₃ |
| 3 | H | —CH₃ | —CH₃ | —CH₂CH═CH₂ | —CH₃ |
| 4 | H | —CH₃ | —CH₂CH═CH₂ | —CH₂CH═CH₂ | —CH₃ |
| 5 | H | —CH₃ | —CH₃ | —CH₂CH═CH₂ | —CH₂CH═CH₂ |
| 6 | H | —CH₃ | —CH₂CH═CH₂ | —CH₃ | —CH₂CH═CH₂ |
| 7 | H | —CH₃ | —CH₂CH═CH₂ | —CH₂CH═CH₂ | —CH₂CH═CH₂ |
| 8 | H | —CH₃ | —CH₃ | —CH₃ | —CH₂CH₂OC(O)CH═CH₂ |
| | | | | | 2-(acryl)ethylene |
| 9 | H | —CH₃ | —CH₃ | 2-(acryl)ethylene | —CH₃ |
| 10 | H | —CH₃ | 2-(acryl)ethylene | 2-(acryl)ethylene | —CH₃ |
| 11 | H | —CH₃ | Allyl | Acrylate | —CH₃ |
| 12 | H | —CH₃ | —(CH₂)ₙN═C═O | —(CH₂)ₙN═C═S | —CH₃ |
| | | | where n = 2-12 | where n = 2-12 | |
| 13 | H | —CH₃ | —(CH₂)ₙN═C═O | —(CH₂)ₙN═C═O | —CH₃ |
| | | | where n = 2-12 | where n = 2-12 | |
| 14 | H | —CH₃ | —(CH₂)ₙN═C═S | —(CH₂)ₙN═C═S | —CH₃ |
| | | | where n = 2-12 | where n = 2-12 | |
| 15 | H | —CH₃ | —(CH₂)ₙCO₂Ar_{F} | —(CH₂)ₙCO₂Ar_{F} | —CH₃ |
| | | | where Ar_{F} is penta | where Ar_{F} is penta | |
| | | | or tetrafluorophenyl | or | |
| | | | | tetrafluorophenyl | |
| 16 | H | —CH₃ | —(CH₂)ₙN₃ where n = | —(CH₂)ₙN₃ where n = | —CH₃ |
| | | | 2-12 | 2-12 | |
| 17 | H | —CH₃ | —(CH₂)ₙC≡CH where | —(CH₂)ₙC≡CH | —CH₃ |
| | | | n = 2-12 | where n = 2-12 | |
| 18 | H | —CH₃ | —(CH₂)ₙN═C═O | —(CH₂)ₙN═C═O | —CH₃ |
| | | | where n = 2-12 | where n = 2-12 | |
| 19 | H | —CH₃ | —CH₃ | —(CH₂)ₙN═C═S | —CH₃ |
| | | | | where n = 2-12 | |
| 20 | H | —CH₃ | —CH₃ | —(CH₂)ₙCO₂Ar_{F} | —CH₃ |
| | | | | where Ar_{F} is penta | |
| | | | | or | |
| | | | | tetrafluorophenyl | |
| 21 | H | —CH₃ | —CH₃ | —(CH₂)ₙN₃ where n = | —CH₃ |
| | | | | 2-12 | |
| 22 | H | —CH₃ | —CH₃ | —(CH₂)ₙC≡CH | —CH₃ |
| | | | | where n = 2-12 | |
| 23 | —OMe | —CH₃ | —CH₃ | —CH₃ | —CH₂CH═CH₂ |
| 24 | —OMe | —CH₃ | —CH₂CH═CH₂ | —CH₃ | —CH₃ |
| 25 | —OMe | —CH₃ | —CH₃ | —CH₂CH═CH₂ | —CH₃ |
| 26 | —OMe | —CH₃ | —CH₂CH═CH₂ | —CH₂CH═CH₂ | —CH₃ |
| 27 | —OMe | —CH₃ | —CH₃ | —CH₂CH═CH₂ | —CH₂CH═CH₂ |
| 28 | —OMe | —CH₃ | —CH₂CH═CH₂ | —CH₃ | —CH₂CH═CH₂ |
| 29 | —OMe | —CH₃ | —CH₂CH═CH₂ | —CH₂CH═CH₂ | —CH₂CH═CH₂ |
| 30 | —OMe | —CH₃ | —CH₃ | —CH₃ | —CH₂CH₂OC(O)CH═CH₂ |
| | | | | | 2-(acryl)ethylene |
| 31 | —OMe | —CH₃ | —CH₃ | 2-(acryl)ethylene | —CH₃ |
| 32 | —OMe | —CH₃ | 2-(acryl)ethylene | 2-(acryl)ethylene | —CH₃ |
| 33 | —OMe | —CH₃ | Allyl | Acrylate | —CH₃ |
| 34 | —OMe | —CH₃ | —(CH₂)ₙN═C═O | —(CH₂)ₙN═C═S | —CH₃ |
| | | | where n = 2-12 | where n = 2-12 | |
| 35 | —OMe | —CH₃ | —(CH₂)ₙN═C═O | —(CH₂)ₙN═C═O | —CH₃ |
| | | | where n = 2-12 | where n = 2-12 | |
| 36 | —OMe | —CH₃ | —(CH₂)ₙN═C═S | —(CH₂)ₙN═C═S | —CH₃ |
| | | | where n = 2-12 | where n = 2-12 | |
| 37 | —OMe | —CH₃ | —(CH₂)ₙCO₂Ar_{F} | —(CH₂)ₙCO₂Ar_{F} | —CH₃ |
| | | | where Ar_{F} is penta | where Ar_{F} is penta | |
| | | | or tetrafluorophenyl | or | |
| | | | | tetrafluorophenyl | |
| 38 | —OMe | —CH₃ | —(CH₂)ₙN₃ where n = | —(CH₂)ₙN₃ where n = | —CH₃ |
| | | | 2-12 | 2-12 | |
| 39 | —OMe | —CH₃ | —(CH₂)ₙC≡CH where | —(CH₂)ₙC≡CH | —CH₃ |
| | | | n = 2-12 | where n = 2-12 | |
| 40 | —OMe | —CH₃ | —(CH₂)ₙN═C═O | —(CH₂)ₙN═C═O | —CH₃ |
| | | | where n = 2-12 | where n = 2-12 | |
| 41 | —OMe | —CH₃ | —CH₃ | —(CH₂)ₙN═C═S | —CH₃ |
| | | | | where n = 2-12 | |
| 42 | —OMe | —CH₃ | —CH₃ | —(CH₂)ₙCO₂Ar_{F} | —CH₃ |
| | | | | where Ar_{F} is penta | |
| | | | | or | |
| | | | | tetrafluorophenyl | |
| 43 | —OMe | —CH₃ | —CH₃ | —(CH₂)ₙN₃ where n = | —CH₃ |
| | | | | 2-12 | |
| 44 | —OMe | —CH₃ | —CH₃ | —(CH₂)ₙC≡CH | —CH₃ |
| | | | | where n = 2-12 | |
| 45 | H | —CH₃ | —(CH₂)ₙOC(O)CH═CH₂ | —(CH₂)ₙOC(O)CH═CH₂ | —CH₃ |
| | | | where n = 2-12 | where n = 2-12 | |
| 46 | H | —CH₃ | —CH₃ | —(CH₂)ₙOC(O)CH═CH₂ | —CH₃ |
| | | | | where n = 2-12 | |
| 47 | —OMe | —CH₃ | —(CH₂)ₙOC(O)—CH═CH₂ | —(CH₂)ₙOC(O)—CH═CH₂ | —CH₃ |
| | | | where n = 2-12 | where n = | |
| | | | | 2-12 | |
| 48 | —OMe | —CH₃ | —CH₃ | —(CH₂)ₙOC(O)—CH═CH₂ | —CH₃ |
| | | | | where n = | |
| | | | | 2-12 | |

As is apparent, the above indicators with reactive moieties can be utilized as a reactive monomer so as to be integrated into a polymer matrix. In another embodiment, the reactive moieties can be used to form a covalent bond with a compatible reactive functionality on the polymer. For example, an isocyanate moiety can react with an amine or hydroxyl group present on the polymer such as poly(2-hydroxyethylmethacrylate). This post-treatment process allows for site specific application of the indicator to designated areas of the polymer.

Other indicators suitable for use in this invention are those which produce a signal such as an electromagnetic signal upon change in pH from neutral to acidic. Such indicators are well known in the art and include, by way of example only,

TABLE 2

| Indicator | Representative structure | Example of reactive derivative |
|---|---|---|
| Phenol red | 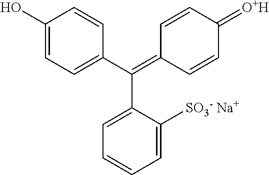 | 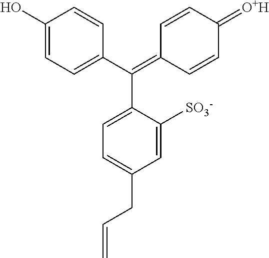 |
| Xylenol Blue | 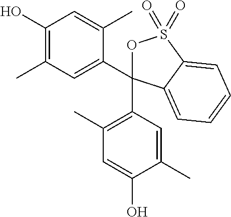 [SMILES: Cc1cc(C2(c3cc(C)c(O)cc3C)OS(=O)(=O)c3ccccc32)c(C)cc1O] | 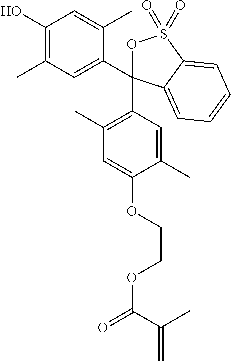 [SMILES: C=C(C)C(=O)OCCOc1cc(C)c(C2(c3cc(C)c(O)cc3C)OS(=O)(=O)c3ccccc32)cc1C] |
| Methyl orange | 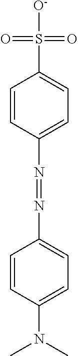 [SMILES: CN(C)c1ccc(N=Nc2ccc(S(=O)(=O)[O-])cc2)cc1] | 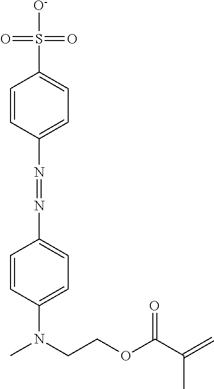 [SMILES: C=C(C)C(=O)OCCN(C)c1ccc(N=Nc2ccc(S(=O)(=O)[O-])cc2)cc1] |

Such reactive compounds can be readily prepared by those skilled in the art.

The term “pharmaceutically acceptable salt” refers to a salt of the compound described herein that is, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein).

The term “signal” refers to any signal that can be detected remotely which signal correlates to the presence of active microbial growth or infection at or adjacent to the site of implantation of the medical device. The signal can be a color change which can be detected by an indicator attached to the medical device and which indicator emits information (typically in the form of readable electromagnetic energy) which can be detected ex vivo. Preferably, the signal is directly in the form of electromagnetic energy which penetrates out of the body and can be ascertained by merely monitoring for that energy.

The term “ex vivo” refers to monitoring or assessment of a signal emitted from the indicator or reporter of the invention located inside the body of a patient using equipment or devices outside the body. That is to say, the signal can be monitored without invasive procedures.

The term “detecting” refers to the use of any device which can determine the presence of a signal. In an embodiment, the signal is monitored continuously such that a machine-readable signal is detected and reported on an on-going basis. In an embodiment, the signal is detected and monitored intermittently, for example periodically every few hours or days. In an embodiment, the signal is detected at discrete times, for example when infection is suspected or when the patient visits a health care facility (e.g., routine check-ups).

The term “electromagnetic energy” refers to any wavelength of energy capable of being transmitted from the body as well as being monitored ex vivo. Examples of such energy include light in the ultraviolet (UV), visible and infrared (IR) portions of the light spectrum. Other examples include energy readable by magnetic resonance imaging (MRI), X-rays, and the like.

The term “produce or can be induced to produce a signal” means that the indicator directly or indirectly produces a signal. An example of indirect production of a signal is the use of energy directed to the indicator to induce fluorescence.

The term “blue light” refers to light that has a wavelength of about 450-500 nm and is more energetic than red light which has a wavelength of about 620 to 750 nm. Blue light penetrates skin well and frequently is used to treat jaundice in newborns by breaking down bilirubin in the blood. In this invention, irradiation of an implanted medical device having covalently bound thereto HMR will allow absorption of the blue light and emittance of fluorescence if there is an active infection.

Alternatively, HMR incorporated into a pH-dependent liposome will be released from degraded liposomes in the presence of acidic pH. That is to say, that microbial growth or an active infection will create an acidic microenvironment which, in turn, will degrade the liposomes and/or alter the structure of HMR into a form that absorbs blue light. Irradiation of the skin area when the implant is made, such as an artificial knee, will indicate infection by virtue of the acidic nature of the microenvironment which can be detected non-invasively by the fluorescence emitted.

The term “pH dependent liposomes” refers to those well-known liposomes which are stable at neutral or alkaline pH but which are unstable under acidic pH conditions. U.S. Patent Pub. No. 2011/0104261 A1, which is incorporated herein by reference in its entirety, discloses pH-sensitive liposomal probes. pH-degradable compositions, including liposomes, are disclosed in U.S. Patent Pub. No. US2013/0064772, and PCT International Patent Pub. No. WO 2013/036771, each of which is hereby incorporated by reference into this application in its entirety.

The term “temperature-sensitive liposomes” refers to those liposomes which are stable at normal body temperature (around 37° C.) but degrade at higher temperatures, such as those present at infection sites. Temperature-sensitive liposomes may be comprised of, for example, dipalmitoylphosphatidylcholine (DPPC) or natural or synthetic thermosensitive polymers. See, for example, Kono and Takagishi, “Temperature-Sensitive Liposomes”, Methods in Enzymology 387, 73-82 (2004).

Liposomes may be comprised of any naturally-occurring or synthetic lipids and/or lipophilic compounds, including, without limitation, phosphatidylcholine, charged lipids (e.g., stearlamine), cholesterol, and/or aminoglycosides. Liposomes, including pH-sensitive liposomes, may also include lipids, lipophilic compounds, and pH-responsive copolymers as described in U.S. Patent Pub. No. 2011/0104261 A1. Liposomes that are sensitive to pH may comprise, for example, a blend of phosphatidylethanolamine (PE), or a derivative thereof, compounds containing an acidic group (e.g., carboxylic group) that acts as stabilizer at neutral pH; pH-sensitive lipids; synthetic fusogenic peptides/proteins; dioleoylphosphatidylethanolamine; and/or attachment of pH-sensitive polymers to liposomes. Use of other compounds, for example distearoylphosphatidylcholine, hydrogenated soya PC, lipid conjugates, phosphatidylethanolamine-poly(ethylene glycol), poly[N-(2-hydroxypropyl)methacrylamide)], poly-N-vinylpyrrolidones, L-amino-acid-based biodegradable polymer-lipid conjugates, or polyvinyl alcohol may allow for decreased leakage of encapsulated compounds and/or longer-lasting liposomes. Similarly, coating surface with inert biocompatible polymers (such as polyethylene glycol, PEG), can increase the longevity of liposomes in vivo while also allowing such polymers to be detached by acidic pH.

In some embodiments, nanoparticles other than lipids may be used to form a delivery vehicle analogous to a liposome. The term “liposome” is meant to encompass such analogous structures.

The term “reporter” refers to compounds that are bound to an antibody or binding fragment thereof and which change at least one of their electromagnetic emission characters when bound to the microbe as compared to that when not bound to the microbe. For example, reporters can be fluorescent indicators which have an altered fluorescence when bound to the microbe as compared to being unbound. For example, the fluorescence signal may be quenched due to the proximity of a quenching molecule in the absence of a microbe; binding of the antibody or fragment to the microbe results in a conformational change such that the quenching molecule is no longer in close enough proximity to exert a quenching effect.

Antibodies, and fragments thereof, that are specific for a variety of infectious bacteria and other microbes are well-known in the art. For example, U.S. Pat. No. 7,531,633 B2 discloses antibodies specific for Staphylococcus aureus. U.S. Patent Application Pub. No. 2013/0022997 discloses antibodies specific for methicillin-resistant Staphylococcus aureus (MRSA) that can distinguish MRSA from methicillin-sensitive Staphylococcus aureus (MSSA). In addition, microbe- and bacteria-specific antibodies are commercially available from a wide variety of vendors, including, for example, Kirkegaard & Perry Laboratories, Inc. and Santa Cruz Biotechnology.

Antibodies (including molecules comprising, or alternatively consisting of, antibody fragments or variants thereof) should have a binding specificity for the microbe(s) of interest such that false positives are avoided. In some embodiments, the antibody or fragment thereof binds to multiple related microbes. In preferred embodiments, the antibodies or fragments thereof specifically bind to an antigen specific for the microbe of interest and do not cross-react with any other antigens.

In an embodiment, the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, for example those described in U.S. Pat. No. 5,939,598 by Kucherlapati et al. An antibody can be humanized, chimeric, recombinant, bispecific, a heteroantibody, a derivative or variant of a polyclonal or monoclonal antibody.

The term “microbe” refers to any infectious organism, including but not limited to a bacterium, fungus, yeast, or virus. Such organisms are well-known in the art. Common infectious bacteria include, but are not limited to, staphylococci, streptococci, Enterococcus faecalis, Escherichia coli, Klebsiella pneumoniae, Proteus mirabilis, and Pseudomonas aeruginosa. Infections are also commonly caused by Candida and mycobacteria.

In some embodiments, the liposome is associated with at least one antibiotic, such as a penicillin, a cephalosporin, a carbapenem, a polymixin, a rifamycin, a lipiarmycin, a quinolone, a sulfonamide, a β-lactam, a fluoroquinolone, a glycopeptide, a ketolide, a lincosamide, a streptogramin, an aminoglycoside, a macrolide, a tetracycline, a cyclic lipopeptide, a glycylcycline, or an oxazolidinone. Antibiotics in these classes are well known in the art. One of ordinary skill in the art would understand that this list is not exhaustive and the use of any antibiotic is within the scope of this invention.

In some embodiments, an anti-infective agent (for example, an antifungal triazole or amphotericin) is associated with the liposome. These may include carbapenems, for example meropenem or imipenem, to broaden the therapeutic effectiveness.

In some embodiments, the indicator comprises a fluorescent moiety, a paramagnetic ion, or a pH sensitive dye which is capable of remote detection. In an embodiment, the indicator is covalently attached to a medical device or to a covering or coating thereof.

In some embodiments, the indicator is associated with one or more liposomes. In some embodiments, the liposomes further comprise an antibody or binding fragment thereof which specifically binds to a microbe and produces a signal indicating the identity of the microbe bound thereto.

In some embodiments, there is provided an antibody or binding fragment thereof has bound thereto a fluorescent moiety which changes its fluorescent character upon binding to the microbe or a change in pH. Such indicators include, by way of example only, 6,7-dihydroxy-4-methylcoumarin and 7-hydroxycoumarin as disclosed above.

In some embodiments, a plurality of different antibodies or binding fragments thereof are bound to the device each producing a unique signal for the microbe bound thereto.

### Conjugation

As necessary or as desired, the indicator or reporter can be conjugated to the surface of the medical device by covalent bonding through compatible functional groups. That is to say that the surface of the medical device contains or is modified to contain a first reactive functional group and the indicator or reporter is modified to contain a compatible functional group. Compatible functional groups are those functionalities which are capable of reacting with the first reactive functional group to form a covalent bond. Non-limiting examples of first reactive functional groups and functional groups compatible therewith are provided in the table below, it being understood that the first reactive functional group and the compatible functional groups can be interchanged. The reactions necessary to form such covalent bonds are well known and are described in numerous standard organic chemistry texts.

| First Reactive | Compatible Functional | Covalent |
|---|---|---|
| Functional Group | Group | bond formed |
| Hydroxyl | Isocyanate | Carbamate |
| Hydroxyl | Chloroformate | Carbonate |
| Hydroxyl | Thioisocyanate | Thiocarbamate |
| Amine | Carboxylic Acid | Amide |
| Amine | Isocyanate | Urea |
| Amine | Thioisocyanate | Thiourea |
| Halo | Phenoxide | Phenylether |

### Detection of Microbial Growth or Infections

Exemplary and non-limiting advantages of the implantable medical devices provided herein include the applicability to any type of implantable medical device. Further advantages include its ability to identify and report the presence of microbial growth or infection adjacent to or on a medical device implanted in a patient. For example, the microbial growth or infection may be detected before the patient presents with the clinical effects of such infection.

In some embodiments, the type of infection can be indicated by the invention. For example, in one embodiment, the medical device has one or more antibodies, or binding fragments thereof, associated therewith. These antibodies are specific for a given bacteria, and when bound to that bacteria produce a unique signal evidencing the presence of the bacteria. In other embodiments, multiple different antibodies or binding fragments thereof can be used, each of which produces a unique signal for the presence of a given strain of bacteria.

In some embodiments, the precise site of microbial growth can be indicated. For example, in one embodiment, the medical device has two or more different indicators and/or reporters attached or incorporated to different locations of the medical device. The signals produced by the different indicators or reporters in response to microbial growth or infection are different, such as fluorescent signals having different wavelengths, and thereby the detection of a signal can be correlated to one of the indicators or reporters, which in turn correlates to the location of the indicator or reporter producing the signal. For example, indicators producing signals with wavelength A under an acidic pH can be incorporated into the inner wall of a catheter while different indicators producing signals with a different wavelength B under the acidic pH can be incorporated into the outer wall of a catheter such that detection of signals with wavelength A indicates microbial growth inside the catheter and detection of signals with wavelength B indicates microbial growth outside the catheter.

### Uses

The implantable medical devices of this invention, in addition to their therapeutic functions (e.g., as a prosthetic joint), are capable of indicating the presence of infection adjacent to or on a medical device implanted in a patient. When an infection is suspected, or as a routine screen to detect microbial growth before clinical signs of infection are present, the desired imaging technology can be used to screen the implantation site for changes in the indicator signal. The device allows early detection and treatment of microbial growth and infection. In some embodiments, the medical device delivers a therapeutic composition to the site of infection. In some embodiments, the patient is treated with antimicrobial compositions, for example orally or intravenously.

In an embodiment, the medical device comprises a high concentration of indicator and/or reporter associated therewith, such that the intensity of the indicator or reporter signal under acidic conditions is high enough to be detectable above the background level of signal, such as that due to chromofluors naturally present in the body. In an embodiment, the signal at the medical device implantation site is determined after implantation but prior to infection. This initial signal intensity can be used as a control for background signal and compared to later signal levels to determine whether the signal has increased or changed, thus indicating the presence of infection.

In another embodiment, the invention also provides for a method of detecting an infection at the surface of a wound. Presently, the clinical diagnosis for an infection of a wound is predicated upon site-specific pain, heat, swelling, discharge, or redness. Though such physiological signals hold a very low predictive value for infection. Unsurprisingly, microbiological analysis from a tissue biopsy is often utilized as an accurate method of confirming an infection in a wound. But this methodology is both invasive and time-consuming, routinely taking between 48 to 72 hours allowing the infection to develop further. The present invention obviates these drawbacks by providing a method whereby instant detection of the infection at the surface of a wound site is facile, safe, and non-invasive. Apropos, the invention is applicable in cases where redness, blood, and/or bruising would obscure typical colorimetric technology used to indicate infection. The present invention utilizes a pH-sensitive fluorescent indicator for such a purpose. The indicator, in the presence of blood and other biological fluids, produces an easily identifiable and readily distinguishable fluorescent signal indicative of microbial growth or an infection on the surface of a wound. This fluorescence can be monitored ex-vivo and is unaffected by bleeding, biological fluid contamination, inflammation, and any other biological obstruction that may occur on the surface of the wound. The present invention also provides early detection of microbial growth, incipient, inapparent, silent, or subclinical infection wherein noticeable symptoms have not developed or will not develop. Microbial growth includes incipient, inapparent, silent, or subclinical infection as a result of microbial growth.

As used herein, the term “surface of a wound” refers to the interface whereupon undamaged tissue is continuous until damaged tissue interrupts the continuum in any given area of the body. Such tissue if not limited to the skin, but can also be ocular or any other part of the integumentary system. It is further contemplated that a wound can comprise more than just the area where the uppermost layer of skin is damaged. Whereas the skin has many layers, a wound may reside in an intermediate layer of dermis located for instance, centimeters below the upper layer of skin. Such injuries and wounds may not be visible to the naked eye and yet the present invention provides for a method of detection wherein a fluorescent signal may indicate infection in this intermediate layer, but also such a signal is detected through the uppermost layer of dermis. In some embodiments, the surface layer of the skin, the epidermis, which is naturally acidic, is wounded or damaged, exposing the inner layers of the skin under the epidermis or even the tissue under the skin which has a physiological pH of above 7 absent any microbial growth. Detection of an acidic pH of the exposed inner layers of the skin or tissue under the skin is an indication of microbial growth. In some embodiments, the wound is due to psoriasis or accidents.

One example of assessing incipient infection in a topical wound would be the skin closure site after surgery where the site is closed, e.g., by sutures. In one embodiment, the sutures can integrate a reporter molecule therein including, for example, covalent bonding. In another embodiment, a bandage can be placed over the closed wound and the bandages interfacing the wound can integrate a reporter molecule. In either event, fluorescence from the reporter molecule arising from generation of an acidic pH is evidence of incipient infection.

In some embodiments, the indicator is dispersed in the material of the device, or a patch that can be placed on the device.

In some embodiments, the indicator is covalently bound to a material of the device. For example, chemically reactive derivatives of fluorescein, such as fluorescein isothiocyanate or carboxyfluorescein succinimidyl ester can react with a functional group, such as amino groups on an antibody or binding fragment thereof, or on a polymer material described herein, or hydroxyl groups present on cellulose (e.g., cotton fiber) or a polymer material (e.g., polyethylene glycol), such that the fluorescent moiety of fluorescein is covalently attached to the material, which can be incorporated into the device. Amino groups can also be introduced to cellulose to react with chemically reactive derivative of fluorescein, such as fluorescein isothiocyanate. See, e.g., Qiang Yang and Xuejun Pan, A facile approach for fabricating fluorescent cellulose, J. Applied Polymer Science, 2010, 117(6): 3639-3644, which is incorporated by reference in its entirety. In some aspects, polyvinyl acetate polymer or a copolymer of polyethylene glycol with polyvinyl acetate can be functionalized by hydrolyzing a percentage (e.g., 0.1% to 10%, such as 0.1%, 0.5%, 1%, 5% or 10%, or any range between any two of the values (end points inclusive)) of the ester groups to an alcohol groups. The alcohol groups can also be converted to other functionalities such as amino groups by methods known in the art. The alcohol or amino groups, or other functional groups may react with the chemically reactive derivatives of fluorescein so that fluorescein moieties are incorporated into the polymer.

The term “fluorescent moiety of fluorescein” refers to the polycyclic chemical moiety that remains after the chemically reactive fluorescein derivative reacts with the functional groups on a material of the device.

In some embodiments, the fluorescent moiety of fluorescein comprises the formula:

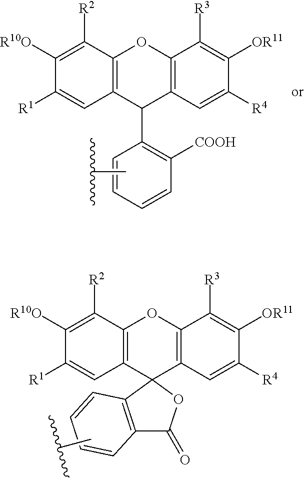

or a tautomer therefore,

wherein
R¹, R², R³ and R⁴ are independently selected from the group consisting of hydrogen, fluoro, chloro, bromo, and —O—C₁-C₄ alkyl,
R¹⁰ and R¹¹ are independently hydrogen or —C(O)C₁-C₄ alkyl; and [symbol] represents the point of connection to the material on the device.

In some embodiments, the chemically reactive fluorescein derivative is a compound of the formula:

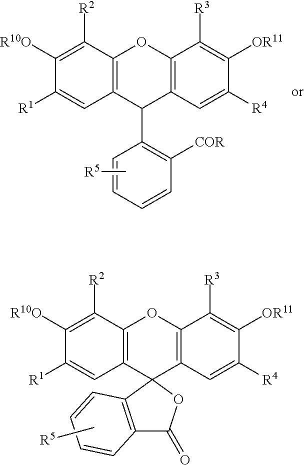

or a tautomer therefore or a salt of the compound or tautomer,
wherein
R¹, R², R³ and R⁴ are independently selected from the group consisting of hydrogen, fluoro, chloro, bromo, and —O—C₁-C₄ alkyl,
R⁵ is selected from the group consisting of C₁-C₄ haloalkyl (C₁-C₄ alkyl substituted with one, two or three chloro, bromo or iodo), —COOR⁶, —NR⁶R⁷, —NHCOR⁶, and —N═C═S;
R is selected from the group consisting of —OH, —O—C₁-C₄ alkyl, and —NR⁸R⁹;
R⁶ is selected from the group consisting of C₁-C₄ haloalkyl, a 5- or 6-membered saturated, unsaturated or aromatic heterocycle ring comprising carbon atoms, one, two or three nitrogen atoms, and zero to one oxygen atom, wherein the heterocycle ring is optionally substituted with one or two substituent selected from the group consisting of oxo (═O), fluoro, chloro and bromo, and —C₁-C₄ alkyl;
R⁷ is hydrogen, or R⁶ and R⁷ together with the nitrogen attached thereto form a 5- or 6-membered saturated heterocycle ring comprising carbon atoms, one or two nitrogen atoms, and zero to one oxygen atom;
R⁸ and R⁹ are independently hydrogen or C₁-C₄ alkyl, or R⁸ and R⁹ together with the nitrogen attached thereto form a 5- or 6-membered saturated heterocycle ring comprising carbon atoms, one or two nitrogen atoms, and zero to one oxygen atom, wherein the heterocycle ring is optionally substituted with a substituent selected from C₁-C₄ alkyl, O—C₁-C₄ alkyl, OH and COOH; and
R¹⁰ and R¹¹ are independently hydrogen or —C(O)C₁-C₄ alkyl.

In some embodiments, R⁵ is —N═C═S.

In some embodiments, R⁵ is

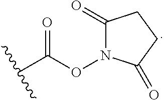 [SMILES: *C(=O)ON1C(=O)CCC1=O]

Examples of chemically reactive fluorescein derivatives include but are not limited to, 5(6)-carboxyfluorescein diacetate N-succinimidyl ester, 5-(bromomethyl)fluorescein, 5-(iodoacetamido)fluorescein, 5-carboxy-fluorescein diacetate N-succinimidyl ester, 5-carboxyfluorescein N-succinimidyl ester, 6-carboxy-fluorescein diacetate N-succinimidyl ester, 6-[fluorescein-5(6)-carboxamido]hexanoic acid N-hydroxysuccinimide ester, 6-carboxy-4′,5′-dichloro-2′,7′-dimethoxyfluorescein N-hydroxysuccinimide ester, fluorescein 5(6)-isothiocyanate, fluorescein diacetate 6-isothiocyanate, and fluorescein isothiocyanate.

In some embodiments, R⁵ is C₁-C₄ bromoalkyl. In some aspects, the C₁-C₄ bromoalkyl can be converted to a C₁-C₄ hydroxyalkyl, for example via hydrolysis under basic conditions. The OH functionality in the C₁-C₄ hydroxyalkyl group react with methanesulfonyl chloride (mesyl chloride, MsCl) under basic conditions (such as in the presence of pyridine) to provide a C₁-C₄ alkyl mesylate. The mesylate functionality in the C₁-C₄ alkyl mesylate group can react with a OH group in a monomer such as hydroxyethylmethacrylate (HEMA) to form a hydroxyethylmethacrylate monomer attached with a fluorescein molecule or a fluorescein derivative. The hydroxyethylmethacrylate monomer attached with a fluorescein molecule or a fluorescein derivative can polymerize with other monomers, such as HEMA or methyl methacrylate (MMA) that do not have a fluorescent indicator attached thereto, to form polymer material that can be used to make the outer surface of a medical device that is capable of indicating the presence or absence of an infection when in contact with a physiological tissue or fluid. Such polymer may comprise 0.1% to 10% of monomers attached with a fluorescent indicator molecule and 90% to 99.9% of monomers that are not attached with a fluorescent indicator molecule. For example, the monomers attached with a fluorescent indicator molecule may be present in 0.1%, 0.5%, 1%, 5% or 10% in the polymer, or any range between any two of the values (end points inclusive).

The polymer material can be made into any desirable shapes or sizes according to its use in the medical device. For example, the polymer may be extruded as pellets, which may be made into a shape in accordance with its use, such as the interior wall of the cartridge of a syringe, or a catheter.

An example for preparing polymers having monomers attached with a fluorescent indicator molecule is illustrated in the following scheme:

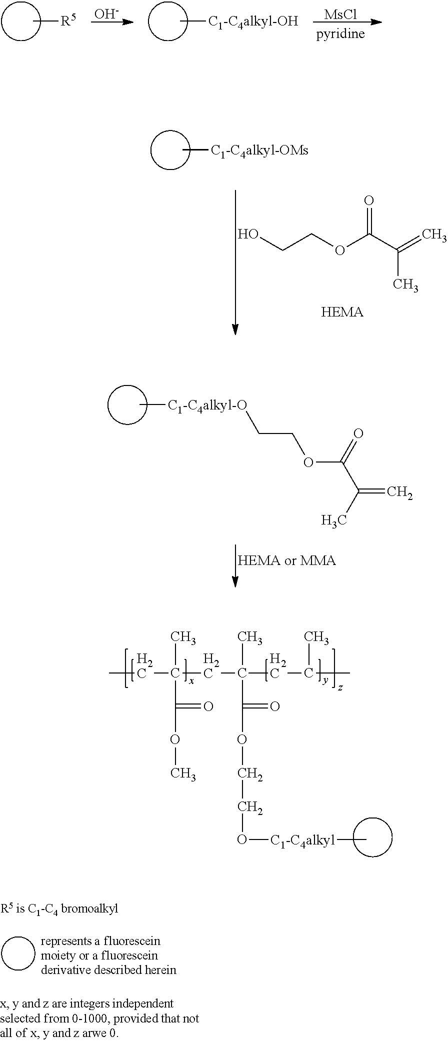

### EXAMPLES

### Example 1

### Preparation of Heptamethoxy Red in Gram Scale

### Step 1: Synthesis of Methyl 2,4,6-trimethoxybenzoate

2,4,6-trimethoxybenzoic acid (5.61 g, 26.42 mmol) was suspended in 20 mL of methanol. Concentrated sulfuric acid (1 mL) was added to the mixture, and the reaction heated to reflux for 24 hrs. The reaction was cooled to room temperature, and the methanol removed in vacuo. The residues were taken up in 50 mL 5% NaHCO₃ and extracted with hexane until all the solids had dissolved. The hexane extract was dried over anhydrous Na₂SO₄, filtered, and the volatiles were removed in a rotary evaporator to dryness to give the desired product, methyl 2,4,6-trimethoxybenzoate, as a white crystalline solid.

### Step 2: Synthesis of Heptamethoxy Red

1-bromo-2,4-dimethoxybenzene (4.23 g, 19.47 mmol) was added to a round bottom flask, and the flask flushed with nitrogen for 10 minutes. Anhydrous ether (80 mL) was added, followed by the drop wise addition of n-butyllithium in hexane (1.6M, 12.2 mL). The cloudy mixture was stirred at room temperature for 10 minutes. Methyl 2,4,6-trimethoxybenzoate (2.20 g, 9.74 mmol) was dissolved in ether, and added drop wise to the reaction mixture. After the addition was complete, the reaction was stirred for 3 minutes longer. The reaction was then poured into a reparatory funnel containing 5% NH₄Cl (50 mL) and shaken until a color change was observed. The layers were separated, and the ether layer was dried over anhydrous Na₂SO₄, filtered, and the volatiles were removed in a rotary evaporator to dryness. The crude oil was placed in the freezer. (Crude yield 6.02 g, 132%).

### Example 2

### One Step Preparation of Heptamethoxy Red

Add (4.23 g, 19.47 mmol) 1-bromo-2,4-dimethoxybenzene to an appropriately sized round bottom flask. Attach a rubber septum to seal the flask. Insert a needle into the septum as a vent and flush the round bottom flask with nitrogen for about 10 minutes. Add (80 mL) anhydrous ether, followed by the drop wise addition of n-butyllithium in hexane (1.6M, 12.2 mL). Stir the cloudy mixture for 10 minutes and keep the round bottom flask on ice. Dissolve (2.20 g, 9.74 mmol) of methyl 2,4,6-trimethoxybenzoate in about 20 ml of anhydrous ether (more than ˜20 mL can be used if needed), and then add this drop wise to the reaction mixture. After the addition is complete, stir the reaction mixture for about 3 minutes longer. Pour the reaction mixture into a separatory funnel containing 5% NH₄Cl (aq) (50 mL) and shake until a color change is observed (pale orange). Allow the layers to separate, and dry the top ether layer with about 5 g anhydrous Na₂SO₄, filter, and remove the volatiles in a rotary evaporator to dryness at 35-40° C. under 400 mbar. Place the crude oil of heptamethoxy red (yellow-orange in color) into the freezer. Yield is ˜3.1 g.

### Example 3

### Preparation of Hexamethoxy Red in Gram Scale

Add (4.23 g, 19.47 mmol) 1-bromo-2,4-dimethoxybenzene to an appropriately sized round bottom flask. Attach a rubber septum to seal the flask. Insert a needle into the septum as a vent and flush the round bottom flask with nitrogen for about 10 minutes. Add anhydrous ether (80 mL), followed by the drop wise addition of n-butyllithium in hexane (1.6M, 12.2 mL). Stir the cloudy mixture for 10 minutes and keep the round bottom flask on ice. Dissolve (2.20 g, 9.74 mmol) of methyl 2,4-dimethoxybenzoate in about 20 ml of anhydrous ether (if needed, more than about 20 ml can be used), and then add this drop wise to the reaction mixture. After the addition is complete, stir the reaction mixture for about 3 minutes longer. Pour the reaction mixture into a separatory funnel containing 5% NH₄Cl (aq) (50 mL) and shake until a color change is observed (pale orange). Allow the layers to separate, and dry the top ether layer with about 5 g anhydrous Na₂SO₄, filter, and remove the volatiles in a rotary evaporator to dryness at 35-40° C. under 400 mbar. Place the crude oil of hexamethoxy red (yellow-orange in color) into the freezer. Yield is about 3.1 g.

### Example 4

Preparation of a Polymerizable Indicator of this Invention

Heptamethoxy red (1 molar equivalent) is heated with an alkyl thiol (1.2-5 molar equivalents) and sodium tertiary butoxide (1.2-5 molar equivalents) in DMF (about 0.5-2 moles/liter with respect to hexamethoxy red). The reaction is monitored for disappearance of hexamethoxy red and/or formation of hydroxylated compounds. When the reaction is substantially complete, the reaction mixture is cooled, Br—(CH₂)ₘ—OC(O)CH═CH₂, where m is 2-10 (preferably in the same molar equivalent as the thiolate), added in situ, and the reaction mixture heated again, if necessary. The polymerizable indicator is isolated from the reaction mixture following aqueous work up and separated by chromatography preferably under neutral to slightly basic conditions, such as by employing neutral or basic alumina, or by employing a slightly alkaline eluent such as an eluent spiked with triethyl amine.

### Example 5

### Detection of Fluorescence

The feasibility of fluorescence as a means to detect incipient microbial growth is dependent upon the ability of the excitation light to penentrate skin and tissue and the ability of the emitted light to penetrate tissue and skin so as to be detectable. In this example, fluorescence was measured in the following manner. A glass surface was coated with fluorescein in a nanogram range on the surface. A chicken breast inclusive of skin, fat and tissue and approximately 1 to 1.5 centimeters in thickness was layered over the glass surface. Excitation light was continuously directed to the surface of the chicken and fluorescence was detected emitting through the chicken breast evidencing that both the excitation light and the emitted light were able to traverse though skin, fat and tissue.

Although the foregoing has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method of determining the presence of microbial growth at or adjacent to a medical device placed on or in a patient which method comprises:
(a) selecting an implantable medical device having on at least part of its surface self-identifying indicators which indicators produce a differential signal under acidic pH as compared to the signal produced at neutral or alkaline pH wherein said signal can be assessed ex vivo;
(b) placing said medical device on or in a patient;
(c) monitoring ex vivo the signal produced by the self-identifying indicators; and
(d) correlating the signal so produced to the presence or absence of microbial growth.

2. The method of claim 1, wherein said indicators are fluorescent indicators which sense pH changes within physiological ranges.

3. The method of claim 1, further comprising:
(e) measuring the signal immediately after implantation to determine a first signal;
(f) measuring the signal at a later time to determine a second signal; and
(g) comparing the first signal and the second signal, wherein a change in signal indicates the presence of the microbial growth.

4. The method of claim 2, further comprising treating the patient with one or more antimicrobial compounds if the presence of the presence of microbial growth is determined.

5. A method to determine the microbe(s) present at or adjacent to a medical device implanted in a patient which method comprises:
(a) selecting an implantable medical device having on at least part of its surface self-identifying reporters which reporters produce a differential signal when bound to a microbe as compared to the signal produced when not bound to the microbe wherein said signal can be assessed ex vivo,
(b) placing said medical device in a patient;
(c) monitoring ex vivo the signal produced by the self-identifying reports; and
(d) correlating the signal so produced to the presence or absence of the microbe at or adjacent to the medical device implanted in the patient.

6. The method of claim 5, further comprising treating the patient with one or more antimicrobial compounds if the presence of the microbe is determined.

7. The method of claim 5, further comprising:
(e) measuring the signal immediately after implantation to determine a first signal;
(f) measuring the signal at a later time to determine a second signal; and
(g) comparing the first signal and the second signal, wherein a change in signal indicates the presence of microbe.

8. The method of claim 7, further comprising treating the patient with one or more antimicrobial compounds if the presence of the microbe is determined.

9. A method of determining the presence of an infection at or adjacent to a medical device placed on or in a patient which method comprises:
(a) selecting a medical device comprise self-identifying indicators which indicators produce a differential signal under acidic pH as compared to the signal produced at neutral or alkaline pH wherein said signal can be assessed ex vivo;
(b) placing said medical device on or in a patient;
(c) monitoring ex vivo the signal produced by the self-identifying indicator; and
(d) correlating the signal so produced to the presence or absence of an active infection.

10. The method of claim 9, wherein the medical device is a topical device.

11. The method of claim 10, wherein said topical device is selected from sutures, bandages, and wraps.

12. The method of claim 11, wherein said sutures are impregnated with the self-identifying indicator.

13. The method of claim 12, wherein said self-identifying indicator is a pH indicator or a pH sensitive fluorescent molecule.

14. The method of claim 13, wherein said self-identifying indicator is fluorescein.
